(19) United States
(12) Patent Application Publication
(10) Pub. No.: US 2008/0021271 A1
(43) Pub. Date: Jan. 24, 2008

(54) Endoscope with a digital view system such as a digital camera

(76) Inventors: Eros Pasero, Torino (IT); Tassilo Meindl, Torino (IT); Marco Omede', Torino (IT); Domenico Moniaci, Torino (IT)

Correspondence Address: PAUL A. FATTIBENE;FATTIBENE & FATTIBENE, 2480 POST ROAD, SOUTHPORT, CT, 06890, US

(21) Appl. No.: 11/786,622

(22) Filed: Apr. 12, 2007

(30) Foreign Application Priority Data
Apr. 21, 2006 (EP) 06008342.5

(51) Int. Cl. A61B 1/04

(52) U.S. Cl. 600/109

(57) ABSTRACT

An endoscope with a digital viewing system, such as a digital camera, with a housing having a longitudinal axis, with a lens attached to the housing and with an electronic image sensor positioned in the interior of the housing for converting a picture delivered through the lens into a digital format, characterized in that the image sensor is movable transverse to or out of the longitudinal axis. The movement of the image sensor facilitates focusing.

### FIELD OF THE INVENTION

The invention concerns an endoscope with a digital view system such as a digital camera, with a housing having a longitudinal axis, with a lens attached to the housing and with an electronic image sensor positioned in the interior of the housing for converting a picture delivered through the lens into a digital format.

### BACKGROUND OF THE INVENTION

In the state of the art, endoscopes are known. Endoscopes with digital cameras are also known. Prior endoscopes normally have a fixed focus, which means that the distance between a lens in the endoscope and the camera system in the endoscope does not change. On the other hand, endoscopes are known where the distance between an image sensor of the camera in the endoscope and the lens of the endoscope differs. The changing of distance can be conducted automatically or manually.

Due to the limitations of common endoscopes without digital imaging, the use of digital cameras in combination with endoscopes have found wide application and improved diagnostic methods.

Digital cameras for endoscopes include a base station such as a personal computer or PC which provides power to a hand piece which is a part of the housing of the endoscope. The hand piece usually comprises an image sensor such as a charge coupled device or CCD or a complimentary metal oxide semi-conductor or CMOS camera, a light source, a video processing micro-processor and user-interfacing such as buttons for snapshot functionality. In order to provide a good image quality optical systems are sometimes used having lenses and mirrors which focus the image on the image sensor. Currently, there has been a shift from the classical CCD based system to a CMOS based system, which simplifies the interfacing to modern personal computers via connection standards such as universal serial bus or USB or wireless connections such as wireless fidelity (WiFi).

Generally, three types of digital cameras are used with endoscopes; fixed focus, manual focus and autofocus cameras. Due to consumer preferences currently, simple manual mechanisms or autofocus cameras are a must. However, compared to commercially available digital cameras, focus mechanisms for cameras used with endoscopes are limited by their small size and placement in the head part of the hand piece, which is extremely critical in dental, rectal and/or vaginal endoscopy. Endoscopes with a fixed focus are pre-configured for certain optical distances, such as for inter-oral cameras for macro pictures of teeth. However, this greatly limits the applicability of these type of cameras for other uses, such as wide fields or complete panorama views.

Manual focus endoscopes provide the opportunity to adjust the lens distance. However, this solution is improvised and not comfortable. The improved mechanical system controls the distance of the lens to an image sensor, such as a circuit, by indirectly moving the lens with a small motor. However, an inter-oral camera space is limited and therefore these systems are not reliable or are mechanically elaborate.

In the state of the art, autofocus endoscopes have cameras, which are fixed vertically, whereby the image is projected using a mirror and also complicated optical system associated with the camera. Endoscopes relevant for this invention, generally have a housings consist of a hand part, which is held in the operator's hand, and a head part, which is introduced into a human or animal body, especially in some orifice. In the head part of these endoscopes a lens is fixed. The lens is in the side wall of the head part. The image taken by the endoscope is of an area perpendicular to the longitudinal axis through the endoscope, especially through the head part. The positioning of a lens in the very tip of the head part is, however, also possible. The right focus can be achieved in this system by either moving the camera or lenses in the optical path. This avoids small sized mechanical systems in the head part, because of the camera, the focusing system can be mounted in a larger hand part, also known as a handle. Further on, autofocus functionality can be achieved in the solution by standard methods, such as direct reflection or indirect image processing algorithms. However, said solutions are quite complex and therefore large and costly.

### SUMMARY OF THE INVENTION

It is therefore an object of the present invention to overcome the drawbacks of the state of the art. In particular, it is an object of the present invention to provide an endoscope characterized by reduced complexity, low costs and improved reliability.

To this end, in the endoscope according to the present invention, instead of moving the lens, a mechanical system is used in order to move the circuit in which the camera is mounted.

In particular, according to the present invention there is provided an endoscope with a digital view system, such as a digital camera, with a housing having a longitudinal axis, with a lens attached to the housing and with an electronic image sensor positioned in the interior of the housing for converting a picture delivered through the lens into a digital format, wherein the image sensor is moveable transverse to the longitudinal axis.

The invention overcomes the drawbacks of the state of the art by a simple and low cost mechanical mechanism. Instead of moving the lens, the mechanical system is used to move the circuit in which the camera is mounted. The movement can be controlled from the handle of the endoscope, where space limitations are not as stringent as in the head part.

It is advantageous to have another embodiment in which the image sensor is moveable perpendicular to the longitudinal axis. Rectangular movements and the necessary guides are easy to manufacture and very precise.

If the image sensor is fixed to a carrier that is parallel to the longitudinal axis, the carrier can be moved resulting in movement of the image sensor. The necessary geometrical interrelationships are therefore more positive.

To be able to focused on a special object, it is advantageous to provide a focus regulation unit or focusing mechanism in the housing, so as to change the distance between the lens and the image sensor.

If the image sensor comprises a circuit, common elements can be used to provide a digital viewing system, such as a digital camera in the endoscope.

If the focus regulation unit allows a skew or a transverse movement of the carrier relative to the lens, the focus can be advantageously easily changed in this embodiment.

As a screw is easy to manufacture and inexpensive to provide, it is advantageous to configure another embodiment in which the focus regulation unit comprises an adjustment element, such as a wheel or a screw, moving the carrier with respect to the housing.

If the endoscope comprises an interface for relaying the data of the image sensor circuit to a processing unit, such as a personal computer or a display, the picture received by the image sensor circuit through the lens in the interior of the body, for example, can be relayed to the interface and then to a personal computer or a display, so that the image can be easily viewed apart from the object which is probed.

To relay the data easily from one place to the other, it is advantageous to provide an interface, which may be a USB connector and/or a wireless communication module.

In a preferred embodiment, if the housing comprises a head part and a hand part, whereby the head and hand parts are preferably positioned consecutively or adjacent each other relative to the longitudinal axis, a long and easy to handle endoscope results.

To be able to enter the endoscope into small opening s or orifices, it is advantageous to provide a head part having a smaller diameter than the hand part, whereby the head part is suitable or adapted to be inserted in orifices of a human or animal body.

If the lens is fixed to the head part to allow pictures to be transmitted in the interior of the housing, an unwanted movement of the lens relative to the image sensor can be avoided, even if pressure is applied to the lens. The focus therefore stays the same.

If the focus regulation unit or focus mechanism is positioned in the hand part, it is advantageous in that that more space can be used for the movement mechanics.

To achieve a simple endoscope that is easy to use, it is advantageous that the screw or the wheel is positioned on the interior of the hand part and is in contact with the carrier, for traversing or moving the carrier relative to the housing of the hand part.

If a support member is positioned in the housing, separately from the carrier, many advantageous embodiments can be achieved.

If the carrier is pivotally attached to the housing a very low cost version of the endoscope is possible.

To avoid distortions, it is advantageous, if the carrier is pivotally attached with an end, distant to the lens, to the interior of the hand part.

In an advantageous embodiment, if the carrier is moveable parallel to the support member, distortions can be totally avoided.

To provide the relaying of the data, it is advantageous, that the interface is attached to the support member and/or an inter-linkage is used to connect the image sensor to the interface.

To get pictures even of dark objects, it is advantageous, that at least one lamp, preferably a LED or even two LED's, is attached together with the lens in one opening of the housing and preferably covered by a flush covering with the housing.

If the covering is of acrylic glass, pictures are still of good quality and injuries in the orifice or opening can be avoided.

In one embodiment the versatility of the endoscope is maximized with a mode switch permitting a choice between an autofocus and a manual focus mode.

### BRIEF DESCRIPTION OF THE DRAWINGS

In the following, a description will be given with reference to the drawings of particular and/or preferred embodiments of the present invention; it has, however, to be appreciated that the present invention is not limited to the embodiments disclosed, but that the embodiments disclosed only relate to particular examples of the present invention, the scope of which is defined by the appended claims. In the drawings, the figures show:

FIG. 1 is a state of the art endoscope with a fixed focus in a schematic side view;

FIG. 2 is a schematic side part view of the head part of the state of the art fixed focus endoscope of FIG. 1;

FIG. 3 is a schematic part view of the head part and a small area of a hand part of an autofocus endoscope of the state of the art;

FIG. 4 is a schematic side view of a first embodiment of the invention, to show the principal;

FIG. 5 is a schematic side view of an endoscope according to the invention with a support member, carrying the image sensor and being pivotally attached to the housing of the endoscope;

FIG. 6 is a schematic side view of another embodiment of the invention;

FIG. 7 is a schematic side view of another embodiment of the invention;

FIG. 8 is a schematic side view of the enlarged detail of the tip portion of FIG. 7;

FIG. 9 is a schematic side view another embodiment of the invention; and

FIG. 10 is a diagram showing information related to image quality.

### DETAILED DESCRIPTION OF THE PREFERRED EMBODIMENTS

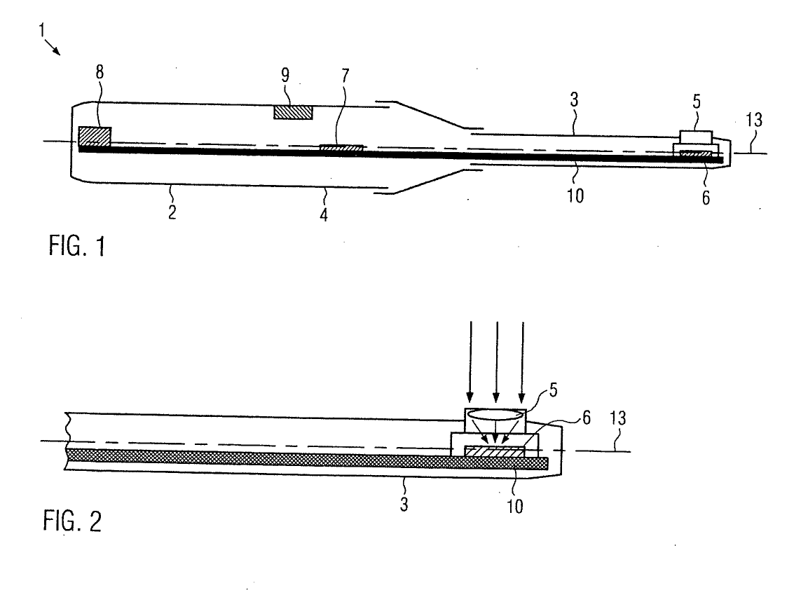

FIG. 1 shows an endoscope 1 with a fixed focus. The endoscope comprises a housing 2. The housing 2 comprises a head part 3 and a hand part 4. The hand part 4, also known as a handle, is the part of the housing 2 of the endoscope 1 which is held by an operator. The head part 3 is entered into an orifice or opening of a human or an animal body. The material used for the housing is preferably of synthetic material. A lens 5 is included in the housing 2, namely in the side of the head part 3, near the tip. The lens 5 is fixed to the head part 3. Through the lens 5, light rays may reach the interior of the housing 2. In the embodiment illustrated in FIG. 1, the light rays are imaged directly on image sensor 6. The image sensor 6 is part of the digital viewing system, such as a digital camera. The light rays relay an image from the outside of the housing 2 and are transformed into electronic information, such as digital information, which is transferred to a video processing unit 7. The video processing unit 7 changes the data and delivers it to an interface 8. The interface 8 may be a USB port or a wireless connection element, such as used for WiFi. In the side of the hand part 3, a button 9 is included, which may be pressed for taking a picture with the endoscope 1.

In the other figures, the same reference numbers are used for the same elements.

In FIG. 2 illustrates the tip area of the head part 3 of the endoscope 1 is shown. As can be seen in FIG. 2 and FIG. 1 the image sensor 6 is mounted on a carrier 10 in the interior of the housing 2. Light rays are imaged by the lens 5 onto the image sensor 6. The state of the art endoscope has a fixed focus, meaning that the distance between the image sensor 6 and the lens 5 cannot be changed. Therefore, an operator has to change the distance between the lens 5 and the object which is viewed.

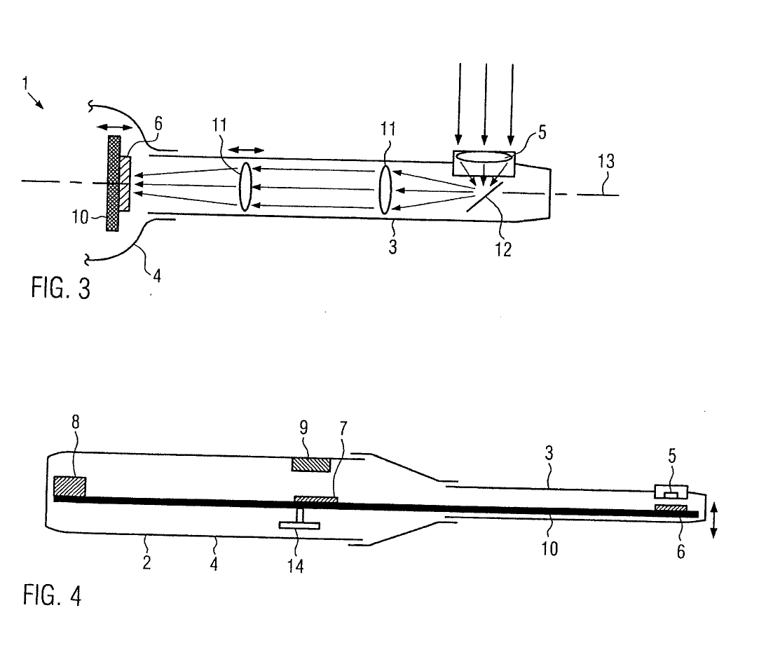

In FIG. 3 illustrates an autofocus endoscope 1, as known from the state of the art, is shown. Apart from the lens 5 in the side of the head part 3, focus lenses 11 are in the interior of the head part 3. The light rays are turned around or folded by use of a mirror 12. The light rays reach the image sensor 6 which is fixed on the carrier 10. The carrier 10 and the fixed image sensor held thereon are movable in a longitudinal direction. In FIGS. 1-3, the longitudinal axis is illustrated having the reference number 13.

FIG. 4 shows the preferred embodiment of the endoscope 1, wherein a CMOS active pixel sensor chip having an analogue output is utilized. Analog to digital conversion related to image processing functions may be implemented in the camera chip. Further, on on-board video compression and processing chip, such as the video processing unit 7, may be provided in order to reduce data to be transmitted and improve image quality onboard.

This significantly reduces space compared to classical CCD based systems. This might be particularly interesting in case of wireless or low-speed interfacing to a base station.

Such a base station may be a personal computer (PC) or a display. Further, the lower power consumption of CMOS image sensor 6 provides for a more efficient power management system in case of a power supply from a battery and wireless data transmission. The invention of this embodiment provides a dental camera in the endoscope 1, having a single lens 5. The image sensor 6 is configured as a circuit on a carrier 10. The circuit is mounted a distance from the single lens 5, in order to focus the image on the camera for extra-oral (infinite distance) images. Positioning the carrier 10 and the circuit closer to the lens 5 provides for the focus of inter-oral and macro images. In this simplest solution the user can perform the positioning of the circuit by turning a wheel of a focus regulation unit or focusing mechanism 14, which is connected to an internal screw, which is fixed towards the circuit and therefore regulates the distance between the camera and the lens 5.

This allows for a very low-cost adjustment of a focus of the inter-oral camera of the endoscope 1.

By moving the wheel of the focus regulation unit 14 the carrier 10 is moved together with the fixed image sensor 6, namely the circuit, is moved, as can be seen at the tip area of the head part 3, towards the lens 5 or away from the lens 5.

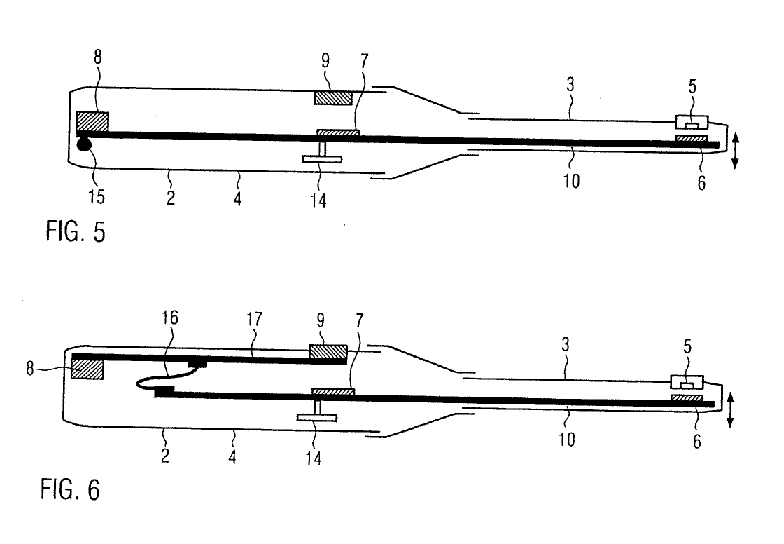

In case of interfacing via a wire, a fixation of the carrier 10 is necessary. The interface also has to be positioned at a certain destination. This can be achieved in one embodiment by fixing the carrier 10 at the housing 2, as can be seen in FIG. 5. Whereas on the one end of the carrier 10 the image sensor 6 is mounted, on the other end the bearing 15 is included. The carrier 10 can pivot around this bearing 15. By pivoting the image sensor 6, it can move away from the lens 5 or towards the lens 5. The movement can be adjusted by the focus regulation unit or focusing mechanism 14. In this case, the positioning may not occur perfectly parallel and therefore an angular distortion of the camera may result. However, if the carrier 10 is long enough, the distortion is insignificant and will not reduce image quality significantly.

Another preferable solution comprises two independent circuits which are connected via a cable 16 in order to prevent the interface unit 8 or the user interface such as a button 9, e.g. a snapshot button, from being moved with the carrier 10, on which the camera is mounted. The button 9 therefore is mounted on the support member 17. Another preferable solution consists in an optimized skew, which allows for an improved positioning of the carrier and the image sensor 6, such as a circuit. In this case, the skew has to be optimized in order to allow movement of e.g. 180 degrees, 270 degrees or 360 degrees, a positioning of the necessary distance in order to achieve focus between e.g. 1 cm and infinite, which might be equal to a real movement of 3 mm of the circuit, dependent on the selected lens 5.

A further preferable solution in order to improve user comfort comprises instead of using an external wheel, a slider is used to controls the positioning of the circuit. The slider can control, for example, the mentioned wheel directly. The focus regulation unit 14 is therefore adjusted. The use of a slider also allows for the attachment of images in order to demonstrate preferred focused positions such as macro, inter oral and extra oral.

In further improved solutions, such preconfigured solutions are realized in the mechanical systems such as by a nose which blocks if the said position is reached. In a further improved solution, mechanical guides are provided which secure the movement in a parallel manner and is performed in a manner that the circuit on which the camera is mounted is moved.

In a further improved solution, the position of the image sensor is performed by a motor and the user can control the positioning indirectly with control buttons.

In a further improved solution, in that the control of the motor can be performed by a microprocessor such as a microcontroller or a FPGA.

In the solution, preconfigured positioning information can be stored in a memory and the user can control by means of, for example, buttons, in order to reach the next step. This allows for comfortable manual focus.

In an alternative solution, the microprocessor elaborates the positioning information from the object distance which can be retrieved by means of measures such as direct reflection measurement.

An alternative solution comprises an image processing algorithm which provides information on the current image quality. The microprocessor then moves the circuit in the position where image quality is best.

Preferably, the user can change the type of focus mechanism between manual and the autofocus mechanism.

Anyhow, all types of mechanical or electromechanical systems are included which allow for positioning of the circuit on which the camera is mounted.

In one embodiment, a personal computer constitutes the base station. In this case, interfacing can be done, for example, via an USB interface. In an alternative embodiment, interfacing is done via wireless interfacing, by means of, for example, WiFi or Bluetooth, but is not limited to any of these standards.

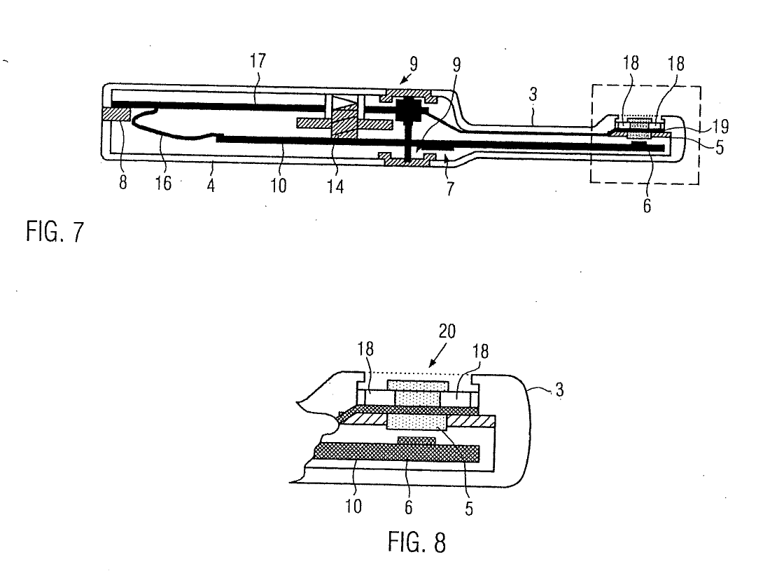

In FIG. 7, illustrates a manual focus embodiment of an endoscope 1 according to the invention with one carrier 10, one support member 17 and a holder are shown. However, more or even less holding devices might be provided in order to achieve the same results. Further on, the mentioned components might be distributed differently on the separate holding devices and inter connection between the holding devices might be different.

The first holding device, namely the support member 17, provides user interfaces such as buttons with, for example, a snap shot function and interfacing to the base station, such as a USB connector or a wireless communication module. On the holding device, power supply circuitry is also mountable. In a preferable embodiment, snap shot buttons are provided. The snap shot buttons are identified with the reference number 9. One snap shot button 9 is on the top side and one is on the bottom side of the housing 2 of the hand part 4. This is in order to allow simple usage.

On the second holding device, namely the carrier 10, the support member 17 which is connected via the cable 16 to the carrier 10, being the first holding device, whereby to the carrier 10 the image sensor 6 is mounted, as well as the image or video processor 7 and associated components. The support member 17 is connected towards the mechanical system that performs the vertical positioning of this holding device. In the preferred embodiment, said mechanical system is an external accessible wheel that turns a screw, which is connected towards the support member 17. Due to the lateral fixation of the support member 17, the rotation maybe transferred into a vertical movement of the carrier 10.

On the third holding device, namely the holder, a number of illumination elements or lamps 18 are mountable, such as white top LEDs. The white top LEDs or lamps 18 provide illumination especially in case of inter or oral usage. The third holding device 19 is connected to the support member 17, in order to receive power for the illumination elements or lamps 18. In order to allow for a regulation of the luminosity of the illumination elements, on the support member 17, a regulator, such as a potentiometer, to change the power provided to said illumination elements may be provide. Said third holding device 19 is mounted in the vicinity of the lens 5. Both, the lens 5 and the illumination elements or lamps 18, are fixed towards the package of the dental camera and the head part 3.

For hygienic reasons, in a preferable solution on top of the illumination elements 18 and the lamps 5, is mounted a transparent element, such as acrylic glass 20 or a glass disk in order to allow simpler cleaning. Acrylic glass or resinous material to be used as a glass substitute is sold under the trademark PLEXIGLAS.

FIG. 8 shows the tip of the head part 3 in more detail.

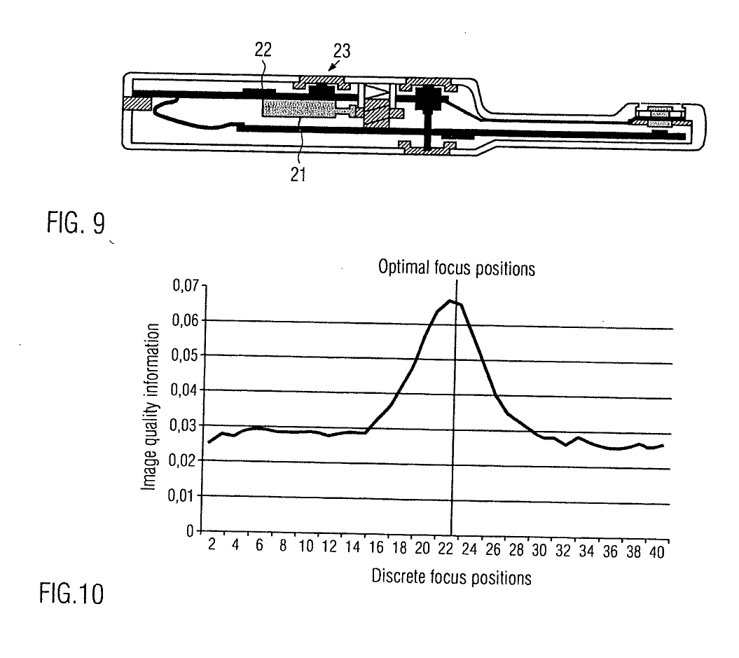

FIG. 9 shows another embodiment of the endoscope 1 according to the invention, with an outer focus modification, having a motor 21, such as a stepper motor, a motor control circuitry 22 and a preferable user interface, such as buttons. A mode switch 23 may also be operated with a button.

By means of this mode switch 23 modes can be selected such as manual focus or auto focus modes. In the manual focus mode the user can control from the user interfaces the desired focus position. The motor control circuitry 22, which might constitute, in the simplest case, power supply circuitry and a micro controller activating the motor, which turns the wheel as described previously in order to control the position of the holding device, namely the carrier 10 and the fixed image sensor 6 placed thereon. In one embodiment a stepper motor may be used which allows precise control as a type of motor 21. A sensor is used which provides information on the exact position of the carrier 10.

The sensor returns information about the position of the wheel which controls the focus, from which the motor control can calculate the actual position of the image sensor 6. In the auto focus mode, the motor control circuitry moves the carrier independently from further user interfaces into the optimal position. The optimal position might be determined from the distance of the object from the lens 5. This distance can be determined, for example, by means of reflection methods known in the art. However, the head part 3 of the dental camera is a small passive solution, which determines from the actual image quality the position in which the carrier 10 has to be moved in order to achieve that image quality. The optimal image quality is, for example, reached by a simple Contrast Detection Method and continuing to move a holding device or carrier until the best contrast is reached.

The optimal focus position relative to the image quality information, shown as discreet focus positions, is illustrated in the graph shown in FIG. 9.

Of course, it should be understood that a wide range of changes and modifications can be made to the embodiments described above without departing from the scope of the present invention. It has to be, therefore, understood that the scope of the present invention is not limited to the embodiments disclosed but is rather defined by the appended claims.

What is claimed is:

1. An endoscope with a digital viewing system, such as a digital camera, comprising:
a housing having a longitudinal axis, a lens attached to the housing;
an image sensor positioned in the interior of the housing for converting a picture delivered through the lens into a digital format; and
wherein said image sensor is moveable transverse to the longitudinal axis.

2. The endoscope, according to claim 1, wherein:
said image sensor is movable perpendicular to the longitudinal axis.

3. The endoscope, according to claim 1, further comprising:
a carrier parallel to the longitudinal axis, said image sensor being fixed to said carrier.

4. The endoscope, according to claim 1 wherein:
said image sensor comprises a circuit.

5. The endoscope, according to claim 1 further comprising:
a focus regulation unit positioned in said housing, adapted to change the distance between said lens and said image sensor.

6. The endoscope, according to claim 5 wherein:
said focus regulation unit allows a skew of said carrier relative to the lens.

7. The endoscope, according to claim 5 further comprising:
a carrier having said image sensor attached at one end; and
said focus regulation unit comprises an adjustment element, adapted to move said carrier relative to said housing.

8. The endoscope, according to claim 1 further comprising:
an interface; and
a processing unit,
whereby said interface is capable of relaying data from said image sensor to said processing unit.

9. The endoscope, according to claim 8, further comprising:
a wireless communication module coupled to said interface.

10. The endoscope, according to claim 1 wherein:
said housing comprises a head part and a hand part,
whereby the hand and head parts are preferably positioned consecutively relative to the longitudinal axis.

11. The endoscope, according to claim 10, wherein:
the head part has a smaller diameter than the hand part,
whereby the head part is adapted to be inserted into orifices of a human or animal body.

12. The endoscope, according to claim 10, wherein:
said lens is fixed to the head part,
whereby pictures are transmitted in the interior of the housing.

13. The endoscope, according claim 10 wherein:
said focus regulation unit is positioned in the hand part.

14. The endoscope, according to claim 7 wherein:
the adjustable element is positioned on the exterior of the hand part and is in contact with said carrier and adapted to traverse said carrier relative to the housing of the hand part.

15. The endoscope, according to claim 1 further comprising:
a support member positioned in said housing, and separate from said carrier.

16. The endoscope, according to claim 3 wherein:
said carrier is pivotally attached to said housing.

17. The endoscope, according to claim 16, wherein:
said carrier is pivotally attached to the interior of the hand part at an end opposite said lens.

18. The endoscope, according to claim 15, wherein:
said carrier is movable parallel to said support member.

19. The endoscope, according to claim 18, wherein:
said interface is attached to said support member and linked to said image sensor.

20. The endoscope, according to claim 1 further comprising:
at least one lamp placed adjacent said lens; and
a flush cover placed over said at least one lamp.

21. The endoscope, according to claim 20, wherein:
said flush cover comprises an acrylic material.

22. The endoscope, according to claim 1 further comprising:
a mode switch adapted to choose between an auto focus and a manual focus mode.

23. An endoscope comprising:
a head part having an opening and a longitudinal axis;
a lens placed in the opening;
a hand part attached to said head part;
a carrier extending into said head part and said hand part substantially parallel to the longitudinal axis;
an image sensor attached at a distal end of said carrier;
a focus regulation unit coupled to said carrier, said focus regulation unit capable of moving said carrier transverse to said longitudinal axis,
whereby said image sensor is capable of selectively moving towards and away from said lens permitting the focusing of an image on said image sensor.

24. An endoscope as in claim 23 wherein:
said carrier is pivotally attached at a proximate end opposite the distal end; and
said focus regulation unit is placed between the proximate end and the distal end of said carrier.

25. An endoscope as in claim 23 wherein:
said focus regulation unit comprises a push button.

26. An endoscope as in claim 23 wherein:
said focus regulation unit comprises a wheel.
